(19) United States
(12) Patent Application Publication
(10) Pub. No.: US 2013/0345116 A1
(43) Pub. Date: Dec. 26, 2013

(54) Composition Containing Trail for Prevention or Treatment of Metabolic Diseases

(71) Applicant: Hye-Jeong LEE, Nam-gu (KR)

(72) Inventor: Hye-Jeong LEE, Nam-gu (KR)

(21) Appl. No.: 13/529,494

(22) Filed: Jun. 21, 2012

(51) Int. Cl. A61K 38/19; A61P 3/00; A61P 9/10; A61P 3/10; A61P 3/06

(52) U.S. Cl. 514/1.9; 514/7.6; 514/6.8; 514/6.9; 514/7.4

(57) ABSTRACT

The present invention provides a pharmaceutical composition and a food composition containing tumor necrosis factor (TNF)-related apoptosis-inducing ligand (TRAIL) as an active ingredient for prevention or treatment of metabolic diseases. The TRAIL according to the present invention can reduce blood glucose, neutral fat, cholesterol, and free fatty acid, and neutral fat in liver, and reduce the synthesis of fat, and promote the lipid metabolism, and thus can be effectively used for the prevention or treatment of metabolic diseases.

### CROSS-REFERENCE TO RELATED APPLICATION

This application claims priority to and the benefit of U.S. Patent Application No. 61/499,353, filed on Jun. 21, 2011, the disclosure of which is incorporated herein by reference in its entirety.

### BACKGROUND

1. Field of the Invention

The present invention relates to a pharmaceutical composition and a food composition containing tumor necrosis factor (TNF)-related apoptosis-inducing ligand (TRAIL) as an active ingredient for prevention or treatment of metabolic diseases.

2. Discussion of Related Art

Obesity is a biological phenomenon caused by the interaction of genetic, metabolic, environmental, and behavioral factors and is generally defined as being overweight. Medically, obesity is defined as having a body mass index (BMI) of 30 or higher (or over 30% of the standard weight) or as having a BMI of 27 or higher and as being associated with other circulatory diseases such as diabetes, hypertension, hyperlipidemia, etc. In particular, obesity is associated with insulin resistance, diabetes, hypertension, cancer, gallbladder disease, hyperlipidemia, arteriosclerosis, etc. and is an important factor in various metabolic diseases and adult diseases. Moreover, it has recently been known that obesity causes a weakened immune system in obesity patients or animals. While obesity is the cause of many diseases, the prevalence of obesity has been steadily increasing, and thus obesity has become an important public health concern. Accordingly, extensive research aimed at developing effective medicines has continued to progress.

Lipid-related metabolic diseases are the diseases associated with blood lipids among the diseases caused by metabolic disorders in vivo and include fatty liver, type 2 diabetes, hyperlipidemia, cardiovascular disease, arteriosclerosis, lipid-related metabolic syndrome, etc. The metabolic syndrome is defined as a clustering of several metabolic diseases such as diabetes, etc. in a patient. Also, there is a need to develop effective medicines for the lipid-related metabolic diseases, similar to obesity.

Diabetes is a chronic metabolic disease, and the incidence of diabetes has also been steadily increasing worldwide. According to statistics, the number of diabetic patients is expected to reach about 220 million people in 2010. Diabetes is generally classified into insulin-dependent diabetes (Type 1 diabetes) and non-insulin-dependent diabetes (Type 2 diabetes), and type 2 diabetes accounts for more than 90% of all diabetic patients. Thus, it is necessary to effectively prevent and treat type 2 diabetes.

Pathogenic mechanism of type 2 diabetes has not been clearly elucidated but is a complex disease that has characteristics of excessive sugar production in liver and insulin resistance in liver, reduced glucose tolerance in muscles and fat cells, etc. In particular, the incidence of type 2 diabetes in overweight people is about 13% higher than normal weight people. Moreover, it is known that obesity is a major factor of diabetes as it is reported that about 85% of type 2 diabetic patients in the USA have obesity. Type 2 diabetes is characterized by both increased peripheral insulin resistance and inadequate insulin secretion. Moreover, type 2 diabetes is a progressive disease and leads to complications such as retinopathy, cataract, nephropathy, neuropathy, atherosclerosis, etc. In order to reduce the occurrence of these complications, it is necessary for type 2 diabetic patients to keep blood sugar levels as close as possible to the normal value, and the treatment for type 2 diabetic patients is focused to optimize and normalize the regulation of blood sugar.

At present, oral drugs for the treatment of type 2 diabetes aimed at regulating blood sugar such as biguanides such as metformin, thiazolidinediones such as pioglytazone and rosiglitazone that activate PPAR γ, α-glucosidase inhibitors that delay intestinal carbohydrate absorption, sulfonylurea that regulate insulin secretion in pancreas β-cells and non-sulfonylurea, etc. are used. However, it has been reported that these drugs have side effects(digestive problem, hepatotoxicity) and thus can cause problems in terms of safety.

Tumor necrosis factor (TNF)-related apoptosis-inducing ligand (TRAIL) is a member of TNF family of cytokines, which exist either as a type II membrane or a soluble protein. TRAIL can induce apoptosis in a wide variety of tumor cells, and thus the well-characterized activity of TRAIL is represented by its anti-cancer activity. However, little is known regarding the effects of TRAIL on cell metabolism, and there is no research on the effects of TRAIL on cell metabolism.

As such, the incidence of metabolic diseases has recently increased, and the occurrence of complications has also become a serious problem. Thus, there is need to develop a new drug or health functional food effective for prevention or treatment of metabolic diseases.

### SUMMARY OF THE INVENTION

The present inventors have studied the effects of TNF-related apoptosis-inducing ligand (TRAIL) on the synthesis of glucose and fat particles, found that TRAIL can reduce the increase in blood glucose, blood fat, and neutral fat in liver, of obesity and type 2 diabetes models induced by high-fat diet, and completed the present invention.

Therefore, an object of the present invention is to provide a composition containing TNF-related apoptosis-inducing ligand (TRAIL) as an active ingredient for prevention or treatment of metabolic diseases.

To achieve the above-described object, the present invention provides a pharmaceutical composition containing TRAIL as an active ingredient for prevention or treatment of metabolic diseases.

Moreover, the present invention provides a food composition containing TRAIL as an active ingredient for prevention or improvement of metabolic diseases.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 is a diagram showing the development of diabetes by the treatment with TRAIL, determined by intraperitoneal glucose tolerance test (IPGTT), in which A: normal dieted GFP-treated group (ND-GFP) and normal dieted TRAIL-treated group (ND-TRAIL), B: high-fat dieted GFP-treated group (HF-GFP) and high-fat dieted TRAIL-treated group (HF-TRAIL), and C: high-fat dieted TRAIL-treated group (HF-TRAIL) and control group, D: conversion into area under the curve (AUC);

FIG. 2 is a diagram showing the effects of TRAIL on the recovery of fatty liver in liver tissue, observed by H&E staining and microscopic examination, in which ND-GFP: normal dieted GFP-treated group, ND-TRAIL: normal dieted TRAIL-treated group, HF-GFP: high-fat dieted GFP-treated group, and HF-TRAIL: high-fat dieted TRAIL-treated group;

FIG. 3 is a diagram showing the change in lipids in liver tissue, observed by Oil Red O staining, in which ND-GFP: normal dieted GFP-treated group, ND-TRAIL: normal dieted TRAIL-treated group, HF-GFP: high-fat dieted GFP-treated group, and HF-TRAIL: high-fat dieted TRAIL-treated group;

FIG. 4 is a diagram showing the change in expression of G6Pase and PEPCK in liver tissue by the treatment with TRAIL, in which ND-GFP: normal dieted GFP-treated group, ND-TRAIL: normal dieted TRAIL-treated group, HF-GFP: high-fat dieted GFP-treated group, and HF-TRAIL: high-fat dieted TRAIL-treated group;

FIG. 5 is a diagram showing the change in expression of Akt protein involved in hepatic lipid metabolism and the change in phosphorylation of Akt protein by the treatment with TRAIL, determined by Western blotting, in which ND-GFP: normal dieted GFP-treated group, ND-TRAIL: normal dieted TRAIL-treated group, HF-GFP: high-fat dieted GFP-treated group, and HF-TRAIL: high-fat dieted TRAIL-treated group; and

FIG. 6 is a diagram showing the change in expression of enzymes and transcription factors involved in the synthesis of fat by treatment with TRAIL, determined by Western blotting, in which ND-GFP: normal dieted GFP-treated group, ND-TRAIL: normal dieted TRAIL-treated group, HF-GFP: high-fat dieted GFP-treated group, and HF-TRAIL: high-fat dieted TRAIL-treated group.

### DETAILED DESCRIPTION OF EXEMPLARY EMBODIMENTS

The present invention provides a composition containing TRAIL as an active ingredient for prevention or treatment of metabolic diseases. The composition comprises a pharmaceutical composition or a food composition.

Hereinafter, the present invention will be described in detail.

Tumor necrosis factor (TNF)-related apoptosis-inducing ligand (TRAIL) according to the present invention reduces blood glucose, blood fat, and neutral fat in the liver. The blood fat may include all types of blood neutral fat, cholesterol, free fatty acid, and the like.

Moreover, the TRAIL reduces the expression of sterol regulatory element binding protein-1c (SREBP-1c). As the expression of SREBP-1c by TRAIL is reduced, the synthesis of fat can be inhibited. The inhibition of the synthesis of fat can be achieved as the expression of enzymes such as fatty acid synthase (FAS), stearoyl CoA desaturase 1 (SCD1), acetyl CoA carboxylase (ACC), and glycerol-3-phosphate acyltransferase (GPAT) is inhibited by TRAIL.

Furthermore, the TRAIL can inhibit the synthesis of blood glucose to effectively prevent or treat metabolic diseases. The inhibition of the synthesis of blood glucose can be achieved by reducing the expression of enzymes involved in the synthesis of glucose such as glucose 6-phosphatase(G6Pase) and phosphoenolpyruvate carboxykinase (PEPCK).

In addition, the TRAIL can promote blood lipid metabolism, and the promotion of lipid metabolism can be achieved by an increase in Akt protein.

As such, the TRAIL according to the present invention can reduce the blood glucose, neutral fat, cholesterol and free fatty acid, and neutral fat in the liver, and reduce the synthesis of fat, and promote the lipid metabolism. Thus, the TRAIL according to the present invention can be effectively used for the prevention or treatment of metabolic diseases.

The “metabolic disease” is defined as a disease group of risk factors for cardiovascular diseases and type 2 diabetes and defined as including insulin resistance, complex and diverse metabolic disorders related thereto, and clinical aspects. In 1988, Reaven stated that the common cause of these symptoms was the insulin resistance associated with impaired in vivo insulin action and suggested the term “insulin resistance syndrome”. However, in 1998, the World Health Organization (WHO) introduced the term “metabolic syndrome” or “metabolic disease” since all the aspects of the symptoms cannot be explained by insulin resistance. Accordingly, the metabolic disease may include all diseases such as non-alcoholic fatty liver diseases, type 2 diabetes, insulin resistance diseases, cardiovascular diseases, arteriosclerosis, lipid-related metabolic disorders, hyperglycemia, hyperinsulinemia, hyperlipidemia, and glucose metabolic disorders.

The non-alcoholic fatty liver is a lipid metabolic disorder of liver and is defined as the excessive accumulation of fat in hepatic cells. The non-alcoholic fatty liver may cause various diseases such as angina pectoris, myocardial infarction, cerebral infarction, arteriosclerosis, fatty liver disease, pancreatitis, etc.

The type 2 diabetes is defined as non-insulin-dependent diabetes that is a chronic disease characterized by relative or absolute deficiency of insulin, resulting in glucose intolerance, and may be caused by insufficient insulin secretion after diet or insulin resistance. The insulin resistance means that the function of insulin is defective and does not stimulate glucose uptake by the cells in the body. If the insulin resistance is high, the body produces too much insulin, which may cause heart diseases, diabetes, etc. as well as hypertension, dyslipidemia, etc. In particular, in the type 2 diabetes, an increase of insulin in muscle and fatty tissues is not perceived, and thus the action of insulin does not occur.

The insulin resistance disease is defined as including diseases caused by the insulin resistance, which means diseases characterized by the resistance of cells to insulin action, hyperinsulinemia, the increase in very-low-density lipoprotein (VLDL) and neutral fat, the decrease in high-density lipoprotein (HDL), hypertension, etc., and is defined as risk factors for cardiovascular diseases and type 2 diabetes (Reaven G M, Diabetes, 37: 1595-607, (1988)). Moreover, it is known that the insulin resistance increases the oxidative stress and changes the signal transduction system in cells together with other risk factors such as hypertension, diabetes, smoking, etc., thus inducing inflammatory responses and leading to atherosclerosis (Freeman BA. et al, Lab Invest. 47: 412-26,(1982)), Kawamura M. et al, J Clin Invest. 94: 771-8, (1994)).

The TRAIL in the present invention comprises various forms of TRAIL, preferably, in can be adenoviral-mediated hTRAIL(Ad.hTRAIL).

The pharmaceutical composition containing TRAIL as an active ingredient for prevention or treatment of metabolic diseases according to the present invention may contain other components within the range that does not impair the advantageous effects of the present invention or may preferably contain other components that have additive or synergistic effects.

Moreover, the pharmaceutical composition of the present invention may further comprise a pharmaceutically acceptable carrier, excipient, and/or diluent in addition to the above-described active ingredients. For example, the carrier, excipient, and/or diluent may be selected from the group consisting of lactose, dextrose, sucrose, sorbitol, mannitol, xylitol, erythritol, maltitol, starch, acacia rubber, alginate, gelatin, calcium phosphate, calcium silicate, cellulose, methylcellulose, microcrystalline cellulose, polyvinylpyrrolidone, water, methyl hydroxybenzoate, propyl hydroxybenzoate, talc, magnesium stearate, and mineral oils.

The pharmaceutical composition of the present invention may be formulated into various dosage forms for oral or parenteral administration by known methods. A typical formulation for parenteral administration may include an injectable formulation and, preferably, be an aqueous isotonic sterile solution or suspension. The injectable formulation may be prepared using a suitable dispersant or wetting agent and a suspending agent by any method known in the art. For example, the respective components may be dissolved in a saline or buffer solution to be formulated into an injectable formulation.

Solid formulations for oral administration include tablets, pills, powders, granules, capsules, etc. The solid formulations may be prepared by mixing the active ingredient with at least one excipient such as starch, calcium carbonate, sucrose, lactose, gelatin, etc. Moreover, the solid formulations may contain, in addition to a simple excipient, a lubricant such as magnesium stearate, talc, etc.

Liquid formulations for oral administration include suspensions, internal solutions, emulsions, syrups, etc. The liquid formulations may include, in addition to commonly used simple diluents such as water and liquid paraffin, various excipients, which are exemplified by humectants, sweeteners, aromatics, preservatives, etc.

Formulations for parenteral administration include sterile aqueous solutions, non-aqueous solutions, suspensions, emulsions, freeze-dried preparations, suppositories, etc. As the non-aqueous solutions and suspensions, propylene glycol, polyethylene glycol, vegetable oils such as olive oil, injectable esters such as ethyl oleate, etc. may be used. As bases of suppositories, witepsol, macrogol, Tween 61, cacao butter, laurin butter, glycerogelatin, etc. may be used.

The effective dosage of the pharmaceutical composition of the present invention may depend on the age, gender, and weight of a patient. However, the pharmaceutical composition of the present invention may be administered in a dose of 1 μg/kg to 100 mg/kg, preferably 10 μg/kg to 10 mg/kg.

Moreover, the food composition according to the present invention may contain the pharmaceutical composition as it is, may be used in conjunction with other foods or food components, and may be appropriately used according to typical methods. Mixed amounts of active ingredients may be suitably determined according to the intended use (preventive, health or therapeutic purposes). Generally, during production of foods or beverages, the composition of the present invention is added in an amount of 15 wt % or less with respect to the total weight of raw materials, preferably, in an amount of 10 wt % or less. However, when prolonged intake is intended for the purpose of health and hygiene or for health control, the amount of the composition may be Smaller than the above range. In addition, there is no problem in terms of safety, and thus the active ingredient of the present invention may also be used in an amount exceeding the above range.

There is no particular limit to kinds of the above-mentioned foods. Examples of foods to which the above material can be added include meat, sausage, bread, chocolate, candy, snack, confectionary, pizza, instant noodles, other noodles, gum, dairy products including ice cream, various soups, beverages, teas, drinks, alcoholic beverages and multi-vitamin preparations. In addition, the above-mentioned food may include any conventional health food.

Similar to conventional beverages, the beverage composition of the present invention may contain various flavoring agents or natural carbohydrates as an additional component. Examples of the natural carbohydrates may include monosaccharides such as glucose and fructose, disaccharides such as maltose and sucrose, polysaccharides such as dextrin and cyclodextrin, synthetic sweetening agents such as saccharin and aspartame, etc. The content of the natural carbohydrate is typically in a range of about 0.01 to 10 g, preferably about 0.01 to 0.1 g per 100 mL of the composition of the present invention.

In addition, the composition of the present invention may further contain various nutritional supplements, vitamins, electrolytes, flavorings, coloring agents, pectic acid and its salts, alginic acid and its salts, organic acids, protective-colloidal thickeners, pH regulators, stabilizers, preservatives, glycerin, alcohols and carbonating agents used in carbonated beverages, etc. Furthermore, the composition of the present invention may contain fruit flesh used for natural fruit juices, fruit juice drinks, and vegetable drinks. These components may be used alone or in combination thereof. The ratio of these additives may not be important, but it is generally selected in the range of 0.01 to 0.1 parts by weight per 100 parts by weight of the composition of the present invention.

The present invention provides a method for treating metabolic diseases, comprising administering to a subject in need thereof an effective amount of tumor necrosis factor (TNF)-related apoptosis-inducing ligand (TRAIL).

The method for treating according to the present invention is useful in the treatment of metabolic diseases by decreasing the concentration of blood glucose, blood neutral fat (plasma TG), blood cholesterol (plasma TC), and blood free fatty acid (plasma FFA), and the fat in liver tissue.

The method for treating according to the present invention can be used alone or in combination with other means of treatment such as surgery, chemotherapy, or radiation therapy.

Next, the present invention will be described in more detail with reference to Examples and Formulation Examples. However, the following Examples and Formulation Examples are provided only for illustrating the present invention, and the scope of the present invention is not limited by the following Examples and Formulation Examples.

### Example 1

### Preparation of Adenovirus Expressing hTRAIL

In order to generate a El-deleted recombinant adenoviral vector expressing human TRAIL (hTRAIL), hTRAIL cDNA was introduced into shuttle plasmid under cytomegalovirus (CMV) transcription-early enhancer/promoter control. El-deleted recombinant adenovirus was engineered to contain only green fluorescent protein (GFP) reporter gene with CMV promoter to form a control group. The recombinant adenovirus was amplified in HEK293 cells and purified by two successive cesium chloride gradient centrifugations.

### Example 2

### Preparation of Obesity and Type 2 Diabetes Models by High-Fat Diet

Obesity and type 2 diabetes models were induced by high-fat diet. Male six-week-old C57BL/6 mice were divided into two groups, which were fed with normal diet (ND) and high-fat diet (HFD: 14.3% protein; 20.1% carbohydrate; and 64.4% fat (kcal %)) for 22 weeks, respectively. Obesity and type 2 diabetes developed in all mice of the high-fat diet group. Adenoviral-mediated hTRAIL (Ad.hTRAIL) or GFP was intravenously administered to the high-fat diet group and normal diet group. The excess expression of Ad.hTRAIL was determined by Western blotting in liver tissue. One week after Ad.hTRAIL and control GFP delivery to each group, the mice were sacrificed and samples were collected to analyze the concentration of blood glucose and insulin, blood neutral fat (plasma TG), blood cholesterol (plasma TC), blood free fatty acid (plasma FFA), and neutral fat in liver tissue(hepatic TG).

The results are shown in Table 1:

TABLE 1

| | Normal diet (ND) | | High-fat diet (HF) | |
|---|---|---|---|---|
| | GFP | TRAIL | GFP | TRAIL |
| Weight(g) | 36.50 ± 0.68 | 36.33 ± 0.79 | 47.00 ± 0.75* | 48.22 ± 0.83* |
| Glucose(mg/dl) | 129.00 ± 3.30 | 116.40 ± 3.75 | 147.60 ± 2.87* | 132.40 ± 3.46† |
| Insulin(ng/dl) | 0.29 ± 0.01 | 0.32 ± 0.01 | 3.67 ± 1.97* | 1.39 ± 0.44 |
| Plasma TG(mg/dl) | 55.02 ± 3.09 | 46.77 ± 1.82 | 104.44 ± 6.99* | 74.33 ± 10.58*† |
| Plasma TC(mg/dl) | 62.07 ± 5.52 | 44.34 ± 2.06 | 135.30 ± 6.35* | 89.20 ± 7.40*† |
| Plasma FFA(uEq/l) | 728.73 ± 34.49 | 851.91 ± 32.91 | 1013.00 ± 34.68* | 767.00 ± 83.81† |
| Hepatic TG(mg/g) | 72.29 ± 5.83 | 53.27 ± 7.69 | 117.62 ± 11.70* | 49.15 ± 2.92† |

P < 0.05 compared with ND GFP-treated mice;
†P < 0.05 compared with HF GFP-treated mice

As shown in Table 1, it was confirmed that were all decreased in the TRAIL-treated group compared to the GFP-treated control group in high-fat diet group. In particular, the neutral fat in liver tissue as well as in blood was also decreased, from which the effects of TRAIL as a drug for treatment of fatty liver could be identified together.

### Example 3

### Recovery of Glucose Tolerance by TRAIL

The development of diabetes was determined by measuring the blood glucose level through intraperitoneal glucose tolerance test (IPGTT). No glucose tolerance means that diabetes is in progress.

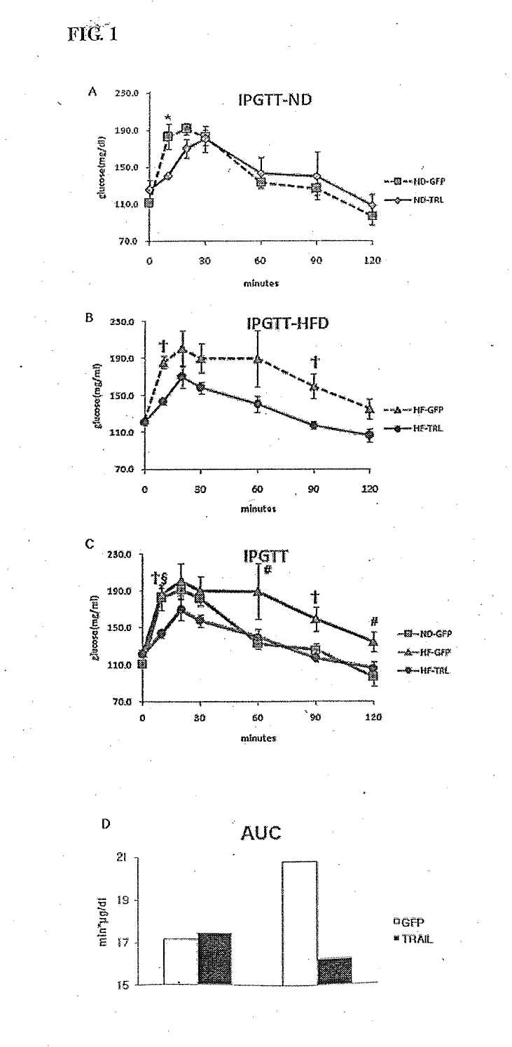

The results are shown in FIG. 1.

As shown in FIG. 1, it could be confirmed that the high-fat dieted GFP-treated control group had obesity and not sugar tolerance, but the high-fat dieted TRAIL-treated group had reduced blood glucose and recovered glucose tolerance, compared to the GFP-treated group. As a result of converting IPGTT values into the area under the curve (AUC), the blood glucose level was very high over time due to impaired glucose tolerance in the GFP-treated group, while the glucose tolerance was recovered to the level of the normal diet group in the TRAIL-treated group, from which the effects of TRAIL as a drug for treatment of type 2 diabetes induced by the high-fat diet were identified.

### Example 4

### Reduction in Fat in Liver Tissue by Treatment with TRAIL

It was determined whether the fatty liver induced by high-fat diet could be recovered by the treatment with TRAIL by identifying the lipid particles of liver tissue thorough histological microscopic examination using H&E staining.

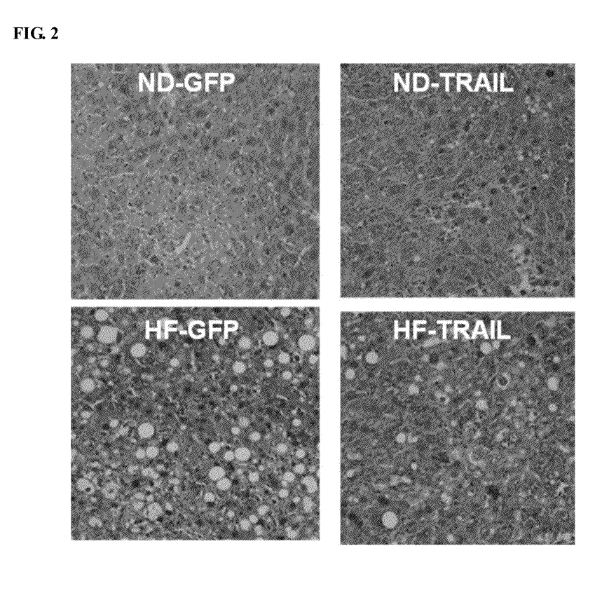

The results are shown in FIG. 2.

As shown in FIG. 2, the hepatic cells in the normal diet and GFP-treated group (ND-GFP) and in the normal dieted TRAIL-treated group (ND-TRAIL) had normal structures, while the livers in the high-fat dieted GFP-treated control group (HF-GFP) had a large amount of lipid particles in liver tissue and showed the presence of fatty liver. However, the livers in the high-fat dieted TRAIL-treated group (HF-TRAIL) had a reduced amount of lipid particles and showed normal liver shapes. As a result, it could be confirmed through the histological microscopic examination that the fatty liver was induced by high-fat diet in the control group and the thus induced fatty liver could be recovered in the TRAIL-treated group.

Moreover, to further identify the effects of improving fatty liver, fat in liver tissue was stained red with Oil Red O staining and subjected to microscopic examination.

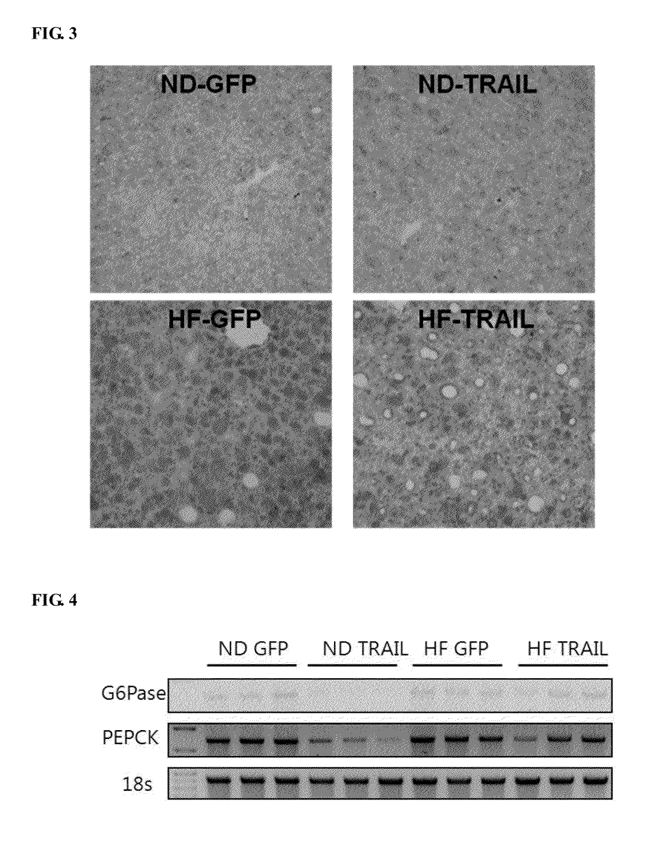

The results are shown in FIG. 3.

As shown in FIG. 3, the red fat particles were rarely observed in both the normal dieted GFP-treated group, and the normal dieted TRAIL-treated group. However, the liver in the high-fat dieted GFP-treated control group (HF-GFP) had a larger amount of red fat particles were observed and showed the presence of fatty liver. However, the red fat particles were decreased in the high-fat dieted TRAIL-treated group. It was determined from these results that the TRAIL could reduce the fat in liver tissue induced by high-fat diet.

### Example 5

### Inhibition of Expression of Glucose Synthetase by TRAIL

High blood glucose level is problematic in metabolic diseases as insulin resistance is a major factor of metabolic diseases. Thus, in order to identify the effects of TRAIL on the expression of glucose 6-phosphatse (G6Pase) and phosphoenolpyruvate carboxykinase (PEPCK) that are important enzymes for the synthesis of blood glucose, the expression of G6Pase and PEPCK genes was analyzed. The results are shown in FIG. 4.

As shown in FIG. 4, the expression of G6Pase and PEPCK genes was decreased in the TRAIL-treated liver in the high-fat dieted TRAIL-treated group (HF-TRAIL), compared to the high-fat dieted GFP-treated control group (HF-GFP). Thus, it was determined from these results that the TRAIL could reduce the blood glucose level by inhibiting the synthesis of glucose.

### Example 6

### Increase in Akt Protein by Treatment of TRAIL

It was determined by Western blotting whether TRAIL protein was effectively expressed in the TRAIL-treated liver and whether the expression of Akt protein involved in the fat metabolism of liver was changed by TRAIL.

The results are shown in FIG. 5.

As shown in FIG. 5, high expression of TRAIL protein was identified in the TRAIL-treated liver. Moreover, the amount of Akt protein involved in the fat metabolism was increased, and the phosphorylation of Akt protein was also increased. Thus, it was determined from these results that the TRAIL could promote the fat metabolism in liver through Akt pathway.

### Example 7

### Inhibition of Synthesis of Fat by Treatment of TRAIL

Sterol regulatory element binding protein-1c (SREBP-1c) is a transcription factor which is synthesized as a precursor in the membranes of the endoplasmic reticulum(ER). SREBP-1c can induce the expression of a family of genes involved in the translocation of glucose and the synthesis of fatty acid. Accordingly, in order to identify whether TRAIL could regulate the expression of SREBP-1c and thus regulate the expression of its target genes of SREBP-1c such as fatty acid synthase (FAS), stearoyl CoA desaturase 1 (SCD1), and acetyl CoA carboxylase (ACC), the change in the expression of enzymes and transcription factors involved in the synthesis of fat in the mice having type 2 diabetes induced by high-fat diet was determined by Western blotting.

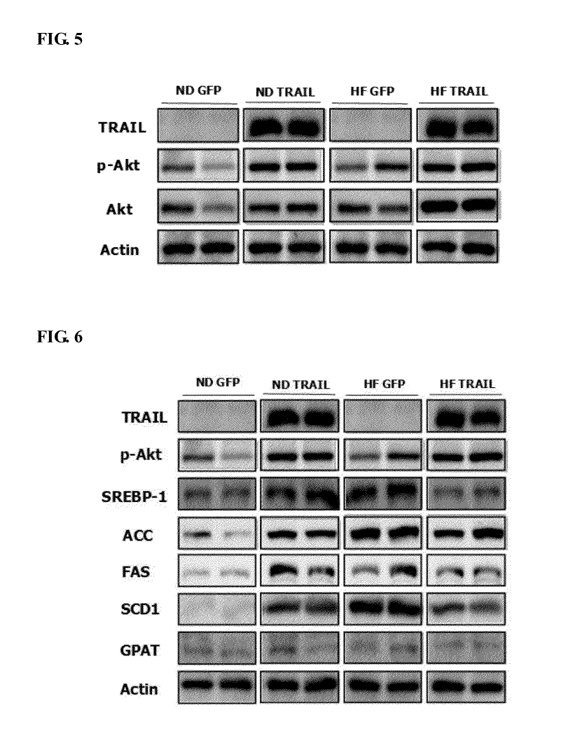

The results are shown in FIG. 6.

As shown in FIG. 6, in the high-fat diet mice, the expression of SREBP-1c was increased, as expected, by the high ability to produce lipids, compared to the normal diet mice. However, as a result of comparing the expression of SREBP-1c in the livers in the normal dieted GFP-treated control group, and the normal dieted TRAIL-treated group, the expression of SREBP-1c in the livers was rather increased in the TRAIL-treated group. However, in the group having type 2 diabetes induced by high-fat diet, the expression of SREBP-1c was reduced by the treatment with TRAIL, and thus the expression of its target lipogenic genes such as FAS, SCD1, ACC, and GPAT was significantly reduced.

### Formulation Example 1

### Preparation of Pharmaceutical Composition

1.1 Preparation of Powders
TRAIL: 20 mg
Lactose: 100 mg
Talc: 10 mg

The above components are mixed and packed in airtight bags, thus preparing powders.

1.2 Preparation of Tablets
TRAIL: 10 mg
Corn starch: 100 mg
Lactose: 100 mg
Magnesium stearate: 2 mg

The above components are mixed and compressed into tablets by a typical tablet preparation method, thus preparing tablets.

1.3 Preparation of Capsules
TRAIL: 10 mg
Crystalline cellulose: 3 mg
Lactose: 14.8 mg
Magnesium stearate: 0.2 mg

The above components are mixed and filled in capsules by a typical capsule preparation method, thus preparing capsules.

1.4 Preparation of Injections
TRAIL: 10 mg
Mannitol: 180 mg
Sterile distilled water for injection: 2,974 mg
Na₂HPO₄.H₂O: 26 mg

Injections each containing the above components per 2 ml ampoule are prepared by a typical injection preparation method.

1.5 Preparation of Liquids
TRAIL: 20 mg
Isomerized sugar: 10 g
Mannitol: 5 g
Purified water: Suitable amount

Liquids are prepared by dissolving the above components in purified water, adding lemon flavor, mixing the above components, adding purified water, storing the mixture of 100 ml in an amber bottle, and then sterilizing the bottle.

### Formulation Example 2

### Preparation of Food Composition

2.1 Preparation of Health Foods
TRAIL: 100 mg
Vitamin mixture: Suitable amount
Vitamin A acetate: 70 μg
Vitamin E: 1.0 mg
Vitamin B1: 0.13 mg
Vitamin B2: 0.15 mg
Vitamin B6: 0.5 mg
Vitamin B12: 0.2 μg
Vitamin C: 10 mg
Biotin: 10 μg
Niacinamide: 1.7 mg
Folic acid: 50 μg
Calcium pantothenate: 0.5 mg
Inorganic mixture: Suitable amount
Ferrous sulfate: 1.75 mg
Zinc oxide: 0.82 mg
Magnesium carbonate: 25.3 mg
Monobasic potassium phosphate: 15 mg
Dibasic potassium phosphate: 55 mg
Potassium citrate: 90 mg
Calcium carbonate: 100 mg
Magnesium chloride: 24.8 mg

Although the above mixing ratio of vitamins and minerals is relatively suitable for health foods in an exemplary embodiment, the mixing ratio may be varied in a wide range. The above components are mixed and formed into granules by a typical health food preparation method, and the formed granules are used in the preparation of a health food composition.

2.2 Preparation of Beverages
TRAIL: 100 mg
Vitamin C: 15 g
Vitamin E (powder): 100 g
Ferrous lactate: 19.75 g
Zinc oxide: 3.5 g
Niacinamide: 3.5 g
Vitamin A: 0.2 g
Vitamin B1: 0.25 g
Vitamin B2: 0.3 g
Water: Suitable amount

The above components are mixed and stirred at 85° C. for about 1 hour. The resulting solution is filtered, sealed in a sterilized 2 L container, refrigerated, and then used in the preparation of a health beverage composition.

Although the above mixing ratio is relatively suitable for favorite beverages in an exemplary embodiment, the mixing ratio may be varied in a wide range according to regional and national preferences such as users' demands, countries, purposes, etc.

As described above, the TRAIL according to the present invention can reduce blood glucose, neutral fat, cholesterol, and free fatty acid, and neutral fat in liver, and reduce the synthesis of fat, and promote the lipid metabolism, and thus can be effectively used for the prevention or treatment of metabolic diseases.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

1-13. (canceled)

14. A method for treating non-alcoholic fatty liver disease, comprising reducing neutral fat in liver and inhibiting synthesis of fat through reducing expression of sterol regulatory element binding protein-1c (SREBP-1c) by administering to a subject in need thereof an effective amount of tumor necrosis factor (TNF)-related apoptosis-inducing ligand (TRAIL).

15. The method of claim 14, wherein the TRAIL reduces blood glucose.

16. The method of claim 14, wherein the TRAIL reduces at least one blood fat selected from the group consisting of blood neutral fat, cholesterol, and free fatty acid.

17-18. (canceled)

19. The method of claim 14, wherein the synthesis of fat is inhibited by inhibiting expression of at least one enzyme selected from the group consisting of fatty acid synthase (FAS), stearoyl CoA desaturase 1 (SCD1), acetyl CoA carboxylase (ACC), and glycerol-3-phosphate acyltransferase (GPAT).

20. The method of claim 14, wherein the TRAIL reduces expression of glucose 6-phosphatase (G6Pase) or phosphoenolpyruvate carboxykinase (PEPCK).

21. The method of claim 14, wherein the TRAIL promotes fat metabolism.

22. The method of claim 21, wherein the fat metabolism is promoted by an increase in Akt protein.

23. The method of claim 14, wherein the TRAIL is adenoviral-mediated hTRAIL(Ad.hTRAIL).
